(19) United States
(12) Patent Application Publication
(10) Pub. No.: US 2020/0316208 A1
(43) Pub. Date: Oct. 8, 2020

(54) MECHANICAL PROCESSING OF BIOPOLYMERS

(71) Applicant: SolyPlus Berlin GmbH, Berlin (DE)

(72) Inventors: Richard Dolph ANDERSEN, Berlin (DE); Annette ASSOGBA-ZANDT, Berlin (DE); Tina LAI, Tai Wai (HK); Isa LIETZAU, Berlin (DE); Elena MALTSEVA, Schöneiche (DE); Andreas VOIGT, Berlin (DE)

(21) Appl. No.: 16/753,447

(22) PCT Filed: Oct. 9, 2018
(86) PCT No.: PCT/IB2018/057788

§ 371 Date: Apr. 3, 2020

(30) Foreign Application Priority Data
Oct. 12, 2017 (DE) 10 2017 009 799.2

(51) Int. Cl. A61K 47/36; C08B 37/08; C08J 3/075; A61K 9/00; A61K 9/70; A61K 45/06

(52) U.S. Cl. CPC A61K 47/36; C08B 37/0072; C08J 3/075; C08J 2305/08; A61K 9/7007; A61K 45/06; A61K 9/0092

(57) ABSTRACT

Embodiments described herein generally relate to methods of processing of biopolymers and applications utilizing these biopolymers.

### PRIORITY CLAIM

This PCT International Patent Application herein claims priority to German priority patent application serial number 102017009799.2, filed Oct. 12, 2017, the entire contents of which are incorporated herein in its entirety.

### FIELD OF THE INVENTION

Embodiments described herein generally relate to methods of processing of biopolymers and applications utilizing these biopolymers.

### BACKGROUND

Most therapeutic dosage forms include mixtures of one or more active pharmaceutical ingredients (APIs) with additional components referred to as excipients. APIs are substances which exert a pharmacological effect on a living tissue or organism, whether used for prevention, treatment, or cure of a disease. APIs can occur naturally, be produced synthetically or by recombinant methods, or any combination of these approaches.

Numerous methods have been devised for delivering APIs into living organisms, each with more or less success. Traditional oral therapeutic dosage forms include both solids (tablets, capsules, pills, etc.) and liquids (solutions, suspensions, emulsions, etc.). Parenteral dosage forms include solids and liquids as well as aerosols (administered by inhalers, etc.), injectables (administered with syringes, micro-needle arrays, etc.), topicals (foams, ointments, etc.), and suppositories, among other dosage forms. Although these dosage forms might be effective in delivering low molecular weight APIs, each of these methods suffers from one or more drawbacks, including the lack of bioavailability as well as the inability to completely control either the spatial or the temporal component of the API's distribution when it comes to high molecular weight APIs. These drawbacks are especially challenging for administering biotherapeutics, i.e. pharmaceutically active peptides (e.g. growth factors), proteins (e.g. enzymes, antibodies), oligonucleotides (e.g. RNA, DNA, PNA), hormones and other natural substances or similar synthetic substances, since many of these pharmacologically active biomolecules are at least partially broken down by the digestive tract or in the blood system and are subsequently delivered in suboptimal dosing to the target site.

Therefore, there is an ongoing need for improved drug-delivery methods in life sciences, including but not limited to human and veterinary medicine. One important goal for any new drug-delivery method is to deliver the desired therapeutic agent(s) to a specific place in the body over a specific and controllable period of time, i.e. controlling the delivery of one or more substances to specific organs and tissues in the body with control of the location and release over time. Methods for accomplishing this localized and time controlled delivery are known as controlled-release drug-delivery methods. Delivering APIs to specific organs and tissues in the body offers several potential advantages, including increased patient compliance, extending activity, lowering the required dose, minimizing systemic side effects, and permitting the use of more potent therapeutics. In some cases, controlled-release drug-delivery methods can even allow the administration of therapeutic agents that would otherwise be too toxic or ineffective for use.

There are traditionally five broad types of solid dosage forms for controlled-delivery oral administration: reservoir and matrix diffusive dissolution, osmotic, ion-exchange resins, and prodrugs. For parenterals, most of the above solid dosage forms are available as well as injections (intravenous, intramuscular, etc.), transdermal systems, and implants. Numerous products have been developed for both oral and parenteral administration, including depots, pumps, micro- and nano-particles.

The incorporation of APIs into polymer matrices acting as a core reservoir is one approach for controlling their delivery. Contemporary approaches for formulating such drug-delivery systems are dependent on technological capabilities as well as the specific requirements of the application. For traditional sustained delivery systems there are two main structural approaches: the controlled release by diffusion through a barrier such as shell, coat, or membrane, and the controlled release by the intrinsic local binding strength of the API(s) to the core or to other ingredients in the core reservoir.

Another strategy for controlled delivery of therapeutic agents, especially for delivering biotherapeutics, is their incorporation into polymeric micro- and nano-particles either by covalent or cleavable linkage or by trapping or adsorption inside porous network structures. Various particle architectures can be designed, for instance core/shell structures. Typically one or more APIs are contained either in the core, in the shell, or in both components. Their concentration can vary throughout the respective component in order to modify their release pattern. Although polymeric nano-spheres can be effective in the controlled delivery of APIs, they also suffer from several disadvantages. For example, their small size can allow them to diffuse in and out of the target tissue. The use of intravenous nano-particles may also be limited due to rapid clearance by the reticuloendothelial system or macrophages. Notwithstanding, polymeric micro-spheres remain an important delivery vehicle.

In view of the above, and in view of the several disadvantages of conventional methods and approaches for drug delivery, there is a significant, long-felt and yet unmet need for improving drug-delivery methods and compositions.

### SUMMARY OF REPRESENTATIVE EMBODIMENTS OF THE INVENTION

It is to be understood that the present invention contemplates certain representative methods and formulations, such as for example certain methods and formulations described herein, in which at least one active pharmaceutical ingredient is present.

It is also to be understood that the present invention also contemplates other representative methods, processes and formulations in which no active pharmaceutical ingredients are present or used at any point during the methods or processes, and therefore the present invention also contemplates formulations in which no active pharmaceutical ingredients are present in the final formulations. Therefore, when certain representative methods, processes and formulations are described herein, it is also to be understood that the present invention also contemplates that such methods, processes and formulations can be adapted or modified in an appropriate and suitable manner, as needed or desired, such that no active pharmaceutical ingredients are present or used at any point during the methods or processes, such that no active pharmaceutical ingredients are present in the final formulations.

Therefore it is to be understood that the methods and processes of the present invention, of which several examples are described herein, can be practiced and implemented in such a manner such that including at least one active pharmaceutical ingredient is optional.

According to certain preferred embodiments, the present invention provides numerous methods of manufacturing and utilizing a biopolymeric bulk material which can be used, for example, in various forms for the delivery of one or more active pharmaceutical ingredients, and which provide numerous, significant unexpected advantages and have numerous applications. These various forms are described in more detail herein, along with numerous potential applications.

### BRIEF DESCRIPTION OF THE FIGURES

FIG. 1 depicts a method for manufacturing a biopolymeric bulk material, comprising: providing at least a biopolymer in dry solid form as powder; providing an aqueous solution; optionally providing at least a pharmaceutically active ingredient; mixing the provided ingredients by means of mechanical energy input to substantially homogeneous distribution, to produce a substantially homogeneous wet mass; and kneading the resulting substantially homogeneous wet mass to substantially bulk material consistency.

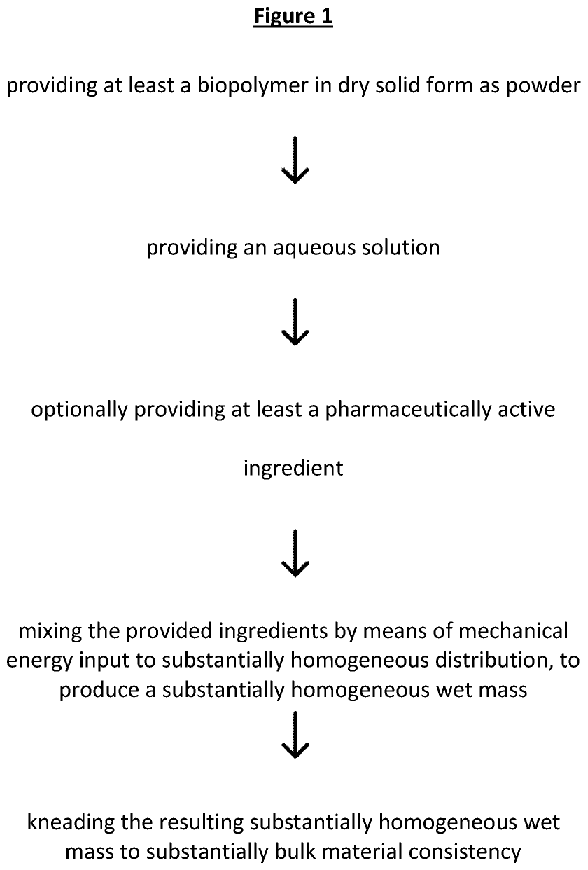

FIG. 2 depicts a method for manufacturing a biopolymeric bulk material, comprising: providing at least a biopolymer microparticle dry powder comprising at least one biopolymer; providing an aqueous solution; optionally providing at least a pharmaceutically active ingredient; mixing the biopolymer and aqueous solution by means of mechanical energy input to substantially homogeneous distribution; and kneading the resulting substantially homogeneous wet mass to substantially bulk material consistency.

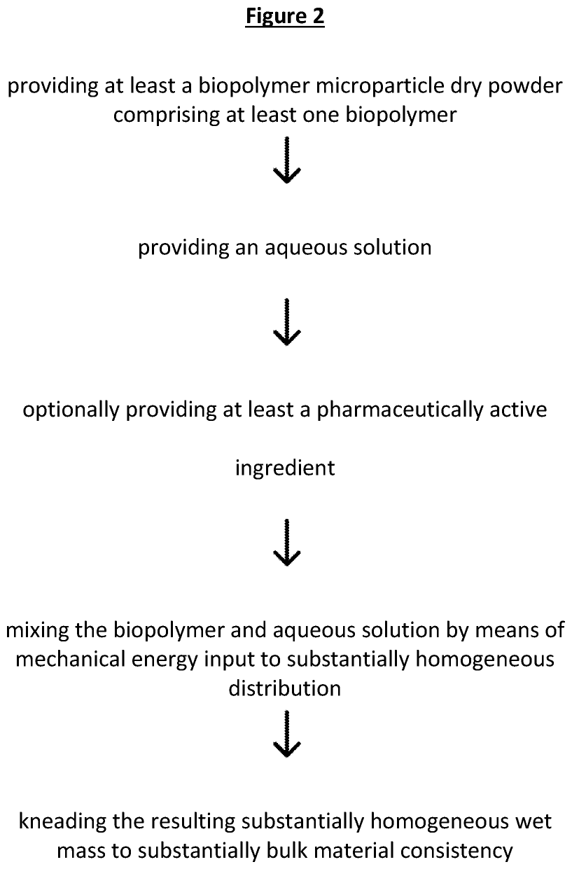

### DETAILED DESCRIPTION OF PREFERRED EMBODIMENTS OF THE INVENTION

Reference will now be made in detail to various aspects of the invention and embodiments. The following language and descriptions of certain preferred embodiments of the present invention are provided to further an understanding of the principles of the present invention. However, it will be understood that no limitations of the present invention are intended, and that further alterations, modifications, and applications of the principles of the present invention are also included.

If not otherwise defined, the term “% w/w” refers to the concentration by weight of a component (e.g. macromolecular compound) based on the total weight of the respective entity (e.g. hydrophilic matrix).

Moreover, unless otherwise defined, all numbers expressing quantities of ingredients, properties such as molecular weight, reaction conditions, and so forth used in the specification are to be understood as being modified in all instances by the term “about.” Accordingly, unless indicated to the contrary, the numerical parameters set forth in the specification are approximations that may vary depending upon the desired and intended properties.

As used herein, the term “substantially” shall be understood to be a definite term that broadly refers to a degree that is, to a significant extent, close to absolute, or essentially absolute. For example, the term “substantially complete” shall be understood to be a definite term that broadly refers to a degree of completeness that is, to a significant extent, close to complete, or essentially complete. In other words, in certain embodiments, and by way of non-limiting example, the term “substantially complete” shall refer to a degree of completeness that is at least about ninety percent or more complete, or that is, to a significant extent, essentially 100 percent complete. For the purpose of this application, if not otherwise stated, particle size is preferably determined microscopically or photographically.

As used herein, the terms “fabricate”, “fabrication” or “fabricating” and “manufacture” or “manufacturing” may be used interchangeably.

Moisture content is preferably determined by formulation and preparation and is preferably determined by a weighing procedure in macroscopic cases.

The present invention provides numerous methods of manufacturing and utilizing a biopolymeric bulk material which can be used, for example, in various forms for the delivery of one or more active pharmaceutical ingredients, and which provide numerous, significant unexpected advantages and have numerous applications. These various forms are described in more detail herein, along with numerous potential applications.

As used herein, it is to be understood that the terms “polymer”, “polymers”, “biopolymer”, “biopolymers” and “biopolymeric” are intended to refer to, but are not limited to, one or more proteins, polysaccharides, carbohydrates, nucleic acids, aptamers, collagen, collagen-n-hydroxysuccinimide, fibrin, gelatin, albumin, alginate, blood plasma proteins, milk proteins, protein-based polymers, hyaluronic acid, chitosan, pectins, gum arabic and other gums, wheat proteins, gluten, starch, cellulose, plant and microorganism cell lysates, copolymers and/or derivatives and/or mixtures and/or chemical modifications of any of said biopolymers, and any combination thereof. In accordance with the methods and applications of the present invention, use of one or more of these polymers or biopolymers results in significant advantages in modifying and improving release characteristics of a drug-delivery composition.

Representative pharmaceutically active compounds or active pharmaceutical ingredients that can be used in accordance with the present invention include, but are not limited to, one or more immunoglobulins, fragments or fractions of immunoglobulins, synthetic substance mimicking immunoglobulins or synthetic, semisynthetic or biosynthetic fragments or fractions thereof, chimeric, humanized or human monoclonal antibodies, Fab fragments, fusion proteins or receptor antagonists (e.g., anti TNF-alpha, Interleukin-1, Interleukin-6 etc.), antiangiogenic compounds (e.g., anti-VEGF, anti-PDGF etc.), intracellular signaling inhibitors (e.g. JAK1,3 and SYK inhibitors) peptides having a molecular mass equal to or higher than 3 kDa, ribonucleic acids (RNA), desoxyribonucleic acids (DNA), plasmids, peptide nucleic acids (PNA), steroids, corticosteroids, an adrenocorticostatic, an antibiotic, an antidepressant, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug an anticholinergic, an antihistaminic, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a haemostatic, a hormone and its synthetic or biosynthetic analogue, an immunosuppressant, an immunostimulant, a mitogen, a physiological or pharmacological inhibitor of mitogens, a mineralcorticoid, a muscle relaxant, a narcotic, a neurotransmitter, a precursor of a neurotransmitter, an oligonucleotide, a peptide, a (para)-sympathicomimetic, a (para)-sympatholytic, a protein, a sedating agent, a spasmolytic, a vasoconstrictor, a vasodilator, a vector, a virus, a virus-like particle, a virustatic, a wound-healing substance, and combinations thereof.

In addition to other methods in which a polymer dry powder (which may be lyophilized) is gradually wetted under and during kneading, the present invention provides for surprisingly advantageous methods in which kneading is separated from wetting. In preferred embodiments, these methods comprise (1) first wetting the polymer (for instance, powder form of lyophilisate or microparticulate powder) in a substantially homogeneous manner by intense vibration/mixing more or less without kneading, and (2) second, kneading the substantially homogeneously wetted polymeric material to provide the material mass for further applications. These novel methods of the present invention have been discovered to have several unexpected advantages.

The methods of the present invention are highly reproducible, in particular because of the use of well-defined starting material, especially well-defined with respect to a starting material that has a much higher degree of wetting homogeneity. It is preferred that the fabrication methods of the present invention begin using dense biomaterial, such as a dense biopolymer, as a starting material.

A preferred starting material for the fabrication methods of the present invention is hyaluronic acid, including for example substantially pure hyaluronic acid. Nonetheless, in addition to the use of hyaluronic acid, it is to be understood that the methods and applications of the present invention, as described herein, can also utilize in a similar manner other biopolymers, mixtures of biopolymers and composites of biopolymers with inorganic or organic matter.

In addition to the many numerous embodiments described herein, other preferred embodiments include improved manufacturing of a hydrophilic matrix or polymeric matrix, including increased quality and efficiency in manufacturing of these matrices.

The present invention also broadly covers methods of manufacturing a drug-delivery composition. In preferred embodiments, a drug-delivery composition comprises at least a hydrophilic matrix or polymeric matrix. By way of non-limiting example, a drug-delivery composition comprises a mixture of at least a hydrophilic matrix or a polymeric matrix and a pharmaceutically active compound.

Further, by way of non-limiting example, a drug-delivery composition comprises at least a hydrophilic matrix, wherein the hydrophilic matrix comprises at least one or more biopolymers, said one or more biopolymers comprising at least one polymer having a molecular weight of at least 10,000 Da, preferably from about 10,000 Da to about four (4) MDa, and more preferably from about 20,000 Da to about two (2) MDa. According to preferred embodiments, suitable biopolymers include but are not limited to chitosan and hyaluronic acid can be used for manufacture of a hydrophilic matrix or polymeric matrix. Other representative biopolymers can include, but are not limited to, one or more of collagen, gelatin, fibrin, or alginate.

Certain representative methods and applications are now described in more detail.

### Manufacturing Example A

According to one preferred embodiment, the present invention provides a method for manufacturing a biopolymeric bulk material, comprising:
providing at least a biopolymer in dry solid form as powder;
providing an aqueous solution;
providing, optionally, at least a pharmaceutically active ingredient;
mixing the provided ingredients by means of mechanical energy input to substantially homogeneous distribution, to produce a substantially homogeneous wet mass; and
kneading the resulting substantially homogeneous wet mass to substantially bulk material consistency.

### Manufacturing Example B

According to another preferred embodiment, the present invention provides a method for manufacturing a biopolymeric bulk material, comprising:
providing at least a biopolymer microparticle dry powder comprising at least one biopolymer;
providing an aqueous solution;
providing, optionally, at least a pharmaceutically active ingredient;
mixing the biopolymer and aqueous solution by means of mechanical energy input to substantially homogeneous distribution; and
kneading the resulting substantially homogeneous wet mass to substantially bulk material consistency.

### Manufacturing Example C

According to yet another preferred embodiment, the present invention provides a method for manufacturing a biopolymeric bulk material containing an active pharmaceutical ingredient, comprising:
providing a biopolymeric bulk material according to “Manufacturing Example A” or “Manufacturing Example 8”;
providing an active pharmaceutical ingredient as powder or solution; and
mixing provided ingredients by means of mechanical energy input to substantial homogeneity.

The present invention also provides novel methods of chemically crosslinking biopolymers, including but not limited to the biopolymers in the biopolymeric bulk material manufactured according to “Manufacturing Example A” or “Manufacturing Example B.”

### Chemical Crosslinking Example A

According to one preferred embodiment, a method of chemically crosslinking biopolymers, including but not limited to the biopolymers in the biopolymeric bulk material manufactured according to “Manufacturing Example A” or “Manufacturing Example 8”, comprises addition of, at least, a chemical crosslinking agent during the steps described in “Manufacturing Example A” or “Manufacturing Example 8”, by dissolving the chemical crosslinking agent into the aqueous solution, or by substituting the aqueous solution partly or completely by the crosslinking agent containing medium. Thereafter, completion of chemical crosslinking can be performed according to any suitable crosslinking protocol.

### Chemical Crosslinking Example B

According to another preferred embodiment, a method of chemically crosslinking the biopolymers, including but not limited to the biopolymers in the biopolymeric bulk material manufactured according to “Manufacturing Example A” or “Manufacturing Example 8”, comprises addition of chemical crosslinking material to the kneaded biopolymeric bulk material. Thereafter, completion of chemical crosslinking can be performed according to any suitable crosslinking protocol.

### Drying Example A

According to yet another preferred embodiment, after manufacturing the biopolymeric bulk material, including but not limited to the biopolymeric bulk material as described in “Manufacturing Example A”, “Manufacturing Example 8” and “Manufacturing Example C”, one or more steps may optionally be performed to substantially or completely dry the biopolymeric bulk materials. In like manner, one or more steps may optionally be performed to substantially or completely dry the biopolymeric bulk materials after chemically crosslinking the biopolymers in the biopolymeric bulk material, including for example the biopolymers described according to “Chemical Crosslinking Example A” or “Chemical Crosslinking Example 8”.

### Manufacturing Example D

According to yet another preferred embodiment, a method for manufacturing a biopolymeric bulk material containing an active pharmaceutical ingredient comprises providing a biopolymeric bulk material according to “Chemical Crosslinking Example A” or “Chemical Crosslinking Example 8”; providing an active pharmaceutical ingredient as a powder or solution; and mixing the ingredients, including the biopolymeric bulk material and the active pharmaceutical ingredient, by means of mechanical energy input to substantial or complete homogeneity.

### Drying Example B

According to yet another preferred embodiment, one or more steps may be performed to substantially or completely dry the crosslinked biopolymeric bulk materials manufactured according to Manufacturing Example D.

### Representative Uses of the Biopolymeric Bulk Materials

According to yet another preferred embodiment, the present invention provides for a variety of uses of biopolymeric bulk materials, including but not limited to the biopolymeric bulk materials described according to any of “Manufacturing Example A”, “Manufacturing Example B”, “Manufacturing Example C”, “Chemical Crosslinking Example A”, “Chemical Crosslinking Example B” or “Manufacturing Example D”. Representative examples include use of the biopolymeric bulk materials for fabrication of applications or for storage under controlled humidity for later usage. The biopolymeric bulk material can also be stored essentially or substantially without loss of its essential and advantageous fabrication rheological properties for months.

According to yet another preferred embodiment, the present invention provides for micronization of the biopolymeric bulk material that is substantially or completely dried, for example as described according to Drying Example A or Drying Example B, by an appropriate cut and mill technology. The micronized biopolymer material may optionally be classified by sieving or a gas/air flow fractionation or any other technology of the art separating solid microparticles under dry conditions. In certain embodiments, the micronized biopolymer particles may optionally be suspended into an oil or into a solvent containing an oil as its main component, to therefore create a suspension. The present invention also provides for a variety of uses of the suspension, including but not limited to uses for pharmaceutical or cosmetic applications; use of the suspension as nose or eye drops; and use of the suspension for topical application to the skin. The present invention also provides for use of the micronized biopolymer particles for inhalative applications targeting the lung epithelium.

### Representative Uses of Biopolymeric Bulk Material for Fabrication of Microneedle Arrays

According to preferred embodiments, the present invention provides improved methods for the fabrication of microneedle arrays. By way of non-limiting example, the present invention provides for use of the biopolymeric bulk material for fabrication of microneedle arrays, wherein this includes but is not limited to use of the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or use of the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months. In preferred embodiments, fabrication of microneedle arrays can be achieved by moulding the biopolymeric bulk material under pressure into mould arrays of any desired geometry (including, but not limited to, needle length, shape and array density) and with any desired shape, size and density and material properties of the microneedles. One or more templates can be used for moulding the biopolymeric bulk material under pressure into mould arrays. In preferred embodiments, after drying, and during moulding under pressure the microneedle arrays are obtained by separation of the template from the microneedle surface-modified biopolymeric bulk material. The microneedle arrays of the present invention are designed and fabricated for a variety of uses and applications, including but not limited to applications in medicine and cosmetics. The microneedle arrays can also be fabricated in such a manner that the microneedle arrays can have any desired geometry (including, but not limited to, needle length, shape and array density) and composition, for instance from pure material to multi-component mixtures. Moreover, the microneedle arrays can be fabricated such that the biopolymeric bulk material can be either substantially or completely dissolvable or undissolvable, and any degree of crosslinking of the biopolymers can be utilized to achieve the desired results during fabrication of the microneedle arrays.

In certain preferred embodiments, moulded microneedle arrays (for example, using a silicon microneedle mould) can be fabricated using pure or substantially pure hyaluronic acid, as well as pure or substantially pure chitosan.

In certain preferred embodiments, the present invention provides for use of the microneedle arrays for transdermal and dermal delivery of one or more pharmaceutical active ingredients.

In still other preferred embodiments, the present invention provides for use of the microneedle arrays for application to the skin by means of a combination of contact pressure and duration. These type of applications to the skin can also be controlled by bandaging techniques.

In still other preferred embodiments, the present invention provides for use of the microneedle arrays for vaccination.

In still other preferred embodiments, the present invention provides for use of the microneedle arrays for intraocular/intravitreal delivery.

In still other preferred embodiments, the present invention provides for use of the microneedle arrays for application to gnat or mosquito bites, itching skin irritations, acne spots, allergic itching spots, itching dermitis spots or local itching skin arrays.

In other preferred embodiments of the present invention, the microneedle arrays consist entirely, or consist essentially, of substantially pure hyaluronic acid or pure hyaluronic acid as the main component.

In still other preferred embodiments, the present invention provides for use of chitosan microneedle arrays or microneedle arrays containing chitosan for application to itching skin arrays.

### Representative Uses of Biopolymeric Bulk Material for Fabrication of Thin and Thick Films

The present invention also provides for use of the biopolymeric bulk material for fabrication of thin and thick films of any shape and size under pressure and subsequent drying, wherein this includes but is not limited to use of the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or use of the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months. In preferred embodiments, the films can be used for any suitable application as a film, or in connection to any number of textile tissues. The films are preferably designed and fabricated for applications in medicine and cosmetics, and for other applications as well that benefit from using thin and thick films. The films can also be designed in any suitable configuration, including but not limited to a plane or foldable or rollable shape or any other desired configuration.

In certain preferred embodiments, the present invention provides for use of the films for covering of internal and topical surfaces, including but not limited to wounds or areas of the skin.

In still other preferred embodiments, the present invention provides for use of the films for topical eye applications.

In still other preferred embodiments, the present invention provides for use of foldable films for application to patients with cystic fibrosis, or for application to body cavities or other conformal coating needs of medical or cosmetic relevance.

### Representative Uses of Biopolymeric Bulk Material for Fabrication of Substantially Solid Bodies

The present invention also provides for use of the biopolymeric bulk material, as described herein, for fabrication of substantially solid bodies of any shape and size, including but not limited to fabrication by means of moulding and mechanical treatment, for instance by utilizing a lathe, by milling, cutting, drilling, and/or piercing. The use of the biopolymeric bulk material, as described herein, for fabrication of the substantially solid bodies can include, but is not limited to, use of the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or use of the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months.

In certain preferred embodiments, these substantially solid bodies are preferably designed and fabricated for a variety of applications in medicine and cosmetics, and for other applications as well that benefit from using the substantially solid bodies.

In still other preferred embodiments, the present invention provides for use of the biopolymeric bulk material, as described herein when the biopolymeric bulk material is used for the fabrication of substantially solid bodies of any shape and size, for medical tools, surgical instruments and accessories, including but not limited to surgical screws, staples, nails, knifes, scissors, sutures, vascular closure devices, etc.

In still other preferred embodiments, the present invention provides for use of the biopolymeric bulk material, as described herein when the biopolymeric bulk material is used for the fabrication of substantially solid bodies of any shape and size, for cosmetic tools and accessories, including but not limited to cosmetic balls, combs, etc.

### Representative Uses of Biopolymeric Bulk Material for Fabrication of Threads or Fibers

In still other preferred embodiments, the present invention provides for use of biopolymeric bulk material for the fabrication of threads or fibers. For example, the threads can be fabricated by means of extrusion, mini-extrusion. For the fabrication of threads or fibers, the use of the biopolymeric bulk material can include, but is not limited to, use of the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or use of the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months. In still other preferred embodiments, the present invention provides for use of the fibers or threads for manufacturing of tissues (e.g., woven or non-woven) from the biopolymeric bulk material described herein, including but not limited to the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D”. In still other preferred embodiments, the present invention provides for use of the tissues (e.g., woven or non-woven) for medical and cosmetic applications.

Representative Uses of Biopolymeric Materials for Fabrication of Porous Materials and/or Solid Foams

In still other preferred embodiments, the present invention provides for the fabrication of porous materials and/or solid foams from the biopolymeric materials described herein, including but not limited to from use of the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or from use of the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months. In a preferred embodiment, the present invention provides for the fabrication of porous materials and/or solid foams from the biopolymeric materials described herein, by inducing an air (or any type of gas)-filled porosity and providing low-density, high-volume biopolymer formulations.

In still other preferred embodiments, the present invention provides for use of the porous materials and/or solid foams for medical and cosmetic applications.

### Representative Uses of Biopolymeric Materials for Fabrication of Inorganic-Organic Hybrid Systems

In still other preferred embodiments, the present invention provides for the fabrication of inorganic-organic hybrid systems comprising composites of biopolymeric materials as described herein. For instance, the biopolymeric materials that can be used for the fabrication of these inorganic-organic hybrid systems include, but are not limited to, the biopolymeric bulk material as described according to any of “Manufacturing Example A”, “Manufacturing Example B”, “Manufacturing Example C”, “Chemical Crosslinking Example A”, “Chemical Crosslinking Example B”, or “Manufacturing Example D” or the biopolymeric bulk material as described elsewhere herein, including biopolymeric bulk material for fabrication of applications or for storage under controlled humidity for later usage, and biopolymeric bulk material that can be stored essentially or substantially without loss of its essential and advantageous fabrication rheological properties for months. These inorganic-organic hybrid systems preferably comprise composites of the biopolymeric materials, as described herein, and inorganic matter, including but not limited to magnetic and electrically conductive materials, pigments, catalytic particles, and/or inorganic micro- and nanoparticles of any kind. The composites can include, for example, electrically conductive composites. In certain embodiments, the present invention provides for use of such electrically conductive composites for manufacturing microneedle arrays.

In still other preferred embodiments, the present invention provides for use of the inorganic-organic hybrid systems, as described herein, for medical devices and cosmetic applications.

### REPRESENTATIVE EXAMPLES

Certain representative, non-limiting examples are shown and described in more detail below. Other embodiments and many of the intended advantages of embodiments will be readily appreciated, as they become better understood by reference to the accompanying detailed description. Those skilled in the art will recognize additional features and advantages upon reading the detailed description which are all within the scope of the invention.

### Example 1—Lyophilized Powder as Starting Material

Ranges of 2-5 (two to five) grams of lyophilized powder of hyaluronic acid (“HA”) Na-salt (can be classified by sieves) (Batch: 041213-E2-P1; 1.64M Dalton; Contipro Biotech) and 1 ml sterilized, unionized water (Millipore; Direct Q-3 UV-R) per gram of HA are put in IKA TUBE MILL C 5000 and grinded with 25,000 rotations per minute for 2 minutes in intervals of 15 seconds with breaks of 1 second. The wetted material then gets kneaded by folding and applying pressure to result in a substantially homogenous mass.

### Example 2—Using Microparticulate Powder as Starting Material

Dry condensed matter (as manufactured in example 1 after micronization) can be classified by sieving with analytical sieves (DIN ISO 3310/1, Apertures of: 80 μm, 53 μm, 25 μm, 20 μm). This can lead to microparticle fractions of greater than 80 μm, 80-53 μm, 53-25 μm, 25-20 μm, and less than 20 μm. These microparticles can be used to produce yet again a kneadable mass which leads to a more homogenous and a more reproducible quality for later applications.

### Example 3—Storage of Already-Formulated Material for Later Usage

The wet starting material (still kneadable) can be stored by raising humidity in a hermetically sealed vial. In this example, cellulose paper was put in a 50 ml falcon tube and wetted to saturation with Millipore water (sterilized, unionized). A cover of a 25 ml falcon tube was then turned around and put atop of the cellulose paper to avoid direct water contact. Different amounts of the kneadable mass can then be stored on top of the second falcon tube cover as long as the whole setup is hermetically sealed to avoid water evaporation.

### Example 4—Moulded Pure Hyaluronic Acid Microneedle Arrays

Ranges of 2-5 (two to five) grams of lyophilized powder of hyaluronic acid (“HA”) Na-salt (can be classified by sieves) (Batch: 041213-E2-P1; 1.64M Dalton; Contipro Biotech) and 1 ml sterilized, unionized water (Millipore; Direct Q-3 UV-R) per gram of HA are put in IKA TUBE MILL C 5000 and grinded with 25,000 rotations per minute for 2 minutes in intervals of 15 seconds with breaks of 1 second. The wetted material then gets kneaded by folding and applying pressure to result in a substantially homogenous mass. The kneaded material is then put into silicon microneedle moulds (Micropoint Technologies Pte Ltd; height 350 μm, base width 150 μm; height 450 μm and 550 μm, base 200 μm; pyramidal microneedles are arranged in a 10×10 square array with 500 μm pitch spacing; the patch size is 8×8 mm). One representative microneedle array section had 350 μm height and 150 μm base dimensions. Another representative microneedle array section had 450 μm height and 200 μm base dimensions. Yet another representative microneedle array section had 550 μm height and 200 μm base dimensions. A piece of gauze bandage was then attached to the upper surface of the still wet material. Pressure can then be applied by hand or devices with an even surface (e.g. glass plates and clamps), and the microneedles can be removed immediately or after drying with/without pressure in the mould. The microneedle arrays can be moulded to have any desired geometries, including but not limited to geometries with respect to length and base.

### Example 5—Moulded Pure Chitosan Microneedle Arrays

One (1) gram of Chitosan (M.W.: 50,000-1,000,000; Chitopharm S; Lot: UPBH0243PR) was ground in IKA TUBE MILL C 5000 with 800 μl of acetic acid (Rotipuran 100%) and 1,200 μl Millipore water (sterilized unionized) with 25,000 rotations per minute for 2 minutes with an interval of 15 seconds and 1 second breaks. The wetted material is then kneaded together forming a substantially homogenous mass. The kneaded material is put into silicon microneedle moulds (Micropoint Technologies Pte Ltd; height 350 μm, base width 150 μm; height 450 μm and 550 μm, base 200 μm; pyramidal microneedles are arranged in a 10×10 square array with 500 μm pitch spacing; the patch size is 8×8 mm). One representative section of a microneedle array had 350 μm height and 150 μm base dimensions. Another representative section of a microneedle array had 450 μm height and 200 μm base dimensions. Yet another representative section of a microneedle array had 550 μm height and 200 μm base dimensions. A piece of gauze bandage was attached to the upper surface of the still wet material. Pressure was applied on the filled mould by 2 glass-plates (5 cm×5 cm×0.6 cm) and a clamp. This whole setup was then dried by air at 60° C. for 24 hours.

In one study, the chitosan microneedles were tested on 4 volunteers with itching mosquito bites. The microneedles were applied multiple times on the same spot by normal pressure and some rubbing movements. All volunteers felt that the application was pleasant. Itching was efficiently stopped after 1-2 minutes and stayed away for a whole day.

### Example 6—Histamine-Containing Hyaluronic Acid Microneedle Array

Histamine dihydrochloride (Lot:WXBC1586V; Sigma-Aldrich) has been soluted in a concentration of 0.3% (m/m) in Millipore water (sterilized, unionized). One (1) ml of this solution was dispersed in one (1) gram of lyophilized hyaluronic acid powder (25 μm-53 μm, classified by analytical sieves) by IKA TUBE MILL C 5000 (25,000 rpm, 2 minutes, 15-seconds interval, 1-second breaks). The wetted material is then kneaded together forming a substantially homogenous mass. The kneaded material is then put into silicon microneedle moulds (Micropoint Technologies Pte Ltd; height in 350 μm, base width 150 μm; height in 450 μm and 550 μm, base in 200 μm; pyramidal microneedles are arranged in a 10×10 square array with 500 μm pitch spacing; the patch size is 8×8 mm). A piece of gauze bandage was attached to the upper surface of the still wet material. Pressure was applied on the filled mould by 2 glass-plates (5 cm×5 cm×0.6 cm) and a clamp. This whole setup was then dried by air at 60° C. for 24 hours. Proof of principle: controlled swelling, reddening and itchy feeling was induced over time (full effect after 10 minutes) by applying the histamine loaded microneedles. No effect was recognized by histamine solution droplet on the skin without microneedle penetration of corneocyte skin layer.

### Example 7—Film/Sheet Manufacturing

In certain embodiments, thin films/sheets of hyaluronic acid can be manufactured preferably by pressing a matrix between glass plates and keeping the pressure up to film/sheet drying. The process can be accelerated by adding wettable textile tissues in intimate contact to films/sheets. The films can be transferred into any type of broken pattern by laser ablation or mechanical action.

In one study, ranges of 2-5 (two to five) grams of lyophilized powder of hyaluronic acid (“HA”) Na-salt (Batch: 041213-E2-P1; 1.64M Dalton; Contipro Biotech) and 1 ml sterilized, unionized water (Millipore; Direct Q-3 UV-R) per gram of HA are put in IKA TUBE MILL C 5000 and grinded with 25,000 rotations per minute for 2 minutes in intervals of 15 seconds with breaks of 1 second. The wetted material then gets kneaded by folding and applying pressure to a substantially homogenous mass. The substantially homogenous kneadable mass is then put between 2 glass-plates (6 cm×6 cm×0.6 cm) Substantially transparent films can also be fabricated in like manner.

Excess material can then be removed as needed or desired to fabricate a finished product.

### Example 8—Oil Suspension

With regard to oil suspensions: micro- and nanoparticles based on the polymer or polymer/drug materials of the present invention are suspended in oil or/an oily composition as a solvent. The oil suspensions are unexpectedly and surprisingly stable with respect to aggregation or coalescence.

In one study, ranges of two to five (2-5) grams of lyophilized powder of hyaluronic acid (“HA”) Na-salt (Batch: 041213-E2-P1; 1.64M Dalton; Contipro Biotech) and 1 ml sterilized, unionized water (Millipore; Direct Q-3 UV-R) per gram of HA are put in IKA TUBE MILL C 5000 and grinded with 25,000 rotations per minute for 2 minutes in intervals of 15 seconds with breaks of 1 second. The wetted material then gets kneaded by folding and applying pressure to produce a substantially homogenous mass. The mass formed this way was then ripped apart to form a bigger surface for drying and dried for 24 h at 60° C. The dry matter was then micronized by usage of IKA TUBE MILL C 5000 (25,000 rpm, 3 minutes, 15 second intervals, 1-second breaks) and classified by analytical sieves (apertures: 106 μm, 80 μm, 53 μm, 25 μm, 20 μm). Ten (10) mg of the fraction of 53 μm-25 μm microparticles was then suspended in 1 ml of Gelo Sitin nose oil (PZN: 03941654; Lot: 243604; containing: sesame oil, dicaprylyl carbonat, orange oil, lemon oil, antioxidant mixture).

In a separate study, with regard to polymer foams or porous bodies, it was surprisingly observed that transfer of polymeric matter (as described herein, in accordance with the present invention) into a foam configuration by dispersion of a gaseous phase into the bulk matter provides a less-dense-than-water material.

### Example 9—Hyaluronic Acid (HA)-Foam with and without Crosslinking

9.1. At first, a crosslinking solution: BDDE (1,4 Butanediol diglycidyl ether 95%; lot:1065835) and acetic acid (Rotipuran; 100%) was mixed in a ratio of 2:1. This solution was then added up with millipore water in a ratio of 1:8. Dispersing this liquid (1 ml of liquid per gram of HA) into lyophilized powder of HA by IKA TUBE MILL C 53000 (25,000 rpm, 2 minutes, 15-second intervals, 1-second breaks) leads to a wet porous (foam) structure. The crosslinking process is then activated by heating to 60° C. for 1 hour hermetically sealed. After the activation the whole setup is dried for 24 h in 60° C.

9.2. Kneadable mass is manufactured in the way stated as in Example 1 (using lyophilized powder as starting product). This kneadable mass was then mixed with 400 mg of dry powder NaHCO₃ by kneading it in. A formed ball of this substance was then dried for 24 h at 60° C. After drying the volume had visibly increased and some fractures on the surface have been noticeable.

### Example 10—Massive Body Formation (e.g. Flowers from Moulds)

In accordance with the present invention, massive bodies can be formed. Macroscopic kneaded and dried material can be exposed to all kinds of shaping and forming, for instance, with a lathe, by milling, cutting, drilling and moulding etc.

10.1. In one study, ranges of 2-5 (two to five) grams of lyophilized powder of hyaluronic acid (“HA”) Na-salt (Batch: 041213-E2-P1; 1.64M Dalton; Contipro Biotech) and 1 ml sterilized, unionized water (Millipore; Direct Q-3 UV-R) per gram of HA are put in IKA TUBE MILL C 5000 and ground with 25,000 rotations per minute for 2 minutes in intervals of 15 seconds with breaks of 1 second. The wetted material is then kneaded by folding and applying pressure to produce a substantially homogenous mass. The kneadable mass can then be moulded in any form by usage of different silicone moulds forming different massive bodies. Moulded bodies could be formed after drying at 60° C. for 24 h., including moulded bodies with a delicate structure of a flower-shaped body.

10.2. Larger batches of the kneadable mass can then be dried for 72 h to evaporate most of the included water in 60° C.

After drying, the raw product can then be drilled, cut, milled or engraved to form various shapes and structures, for example, a screw structure, or different cutting surfaces that can be formed.

### Example 11—Formation of a Filament Structure

Woven tissues, threads and other types of filament structures are manufactured based on the polymer material, such as the dense polymer material (or polymer/polymer or polymer/drug mixtures) of the present invention, such as for example by using mini-extruder action, and these filament structures can be used for braiding, weaving etc.

In one study, ranges of 2-5 (two to five) grams of lyophilized powder of hyaluronic acid (“HA”) Na-salt (Batch: 041213-E2-P1; 1,64M Dalton; Contipro Biotech) and 1 ml sterilized, unionized water (Millipore; Direct Q-3 UV-R) per gram of HA are put in IKA TUBE MILL C 5000 and grinded with 25,000 rotations per minute for 2 minutes in intervals of 15 seconds with breaks of 1 second. The wetted material is then kneaded by folding and applying pressure to produce a substantially homogenous mass. The kneaded mass can then be formed into threads, and the threads are used as a starting material for various filament structures and tissues.

### Example 12—Example of Crosslinking

Chemical crosslinking can be performed, as described further herein in the specification, and all the various applications can be modified by covalent crosslinking for desired control of mechanical, rheological, dissolvable and biodegradable properties.

In one study, a crosslinking solution was first mixed: BDDE (1,4 Butanediol diglycidyl ether 95%; lot:1065835) and acetic acid (Rotipuran; 100%) was mixed in a ratio of 2:1. This solution was then added up with millipore water in a ratio of 1:8. Dispersing this liquid (1 ml of liquid per gram of hyaluronic acid or “HA”) into lyophilized powder of HA by IKA TUBE MILL C 53000 (25,000 rpm, 2 minutes, 15-second intervals, 1-second breaks) leads to a wet porous (foam) structure. The crosslinking process is then activated by heating to 60° C. for 1 hour hermetically sealed. Immediately after dispersing the crosslinking-liquid massive bodies can be formed by moulding under pressure and drying for 24 h in 60° C. In one instance, 1.0600 g body of crosslinked HA was stored for more than 1 month in 25 ml of Millipore water. Equal amounts of non-crosslinked HA would have been dissolved in less than 1 day. No changes in solvent viscosity were observed.

### Example 13—Hyaluronic Acid Microneedles

Scanning electron microscope pictures were used to demonstrate details of hyaluronic acid microneedles that are fabricated in accordance with the present invention. As described elsewhere herein, in certain preferred embodiments, moulded microneedle arrays (for example, using a silicon microneedle mould) can be fabricated using pure or substantially pure hyaluronic acid, as well as pure or substantially pure chitosan.

1. A method for manufacturing a biopolymeric bulk material, comprising:
providing at least one biopolymer in dry solid form as powder;
providing an aqueous solution;
optionally providing at least one pharmaceutically active ingredient;
mixing the provided ingredients by means of mechanical energy input to substantially homogeneous distribution, to produce a substantially homogeneous wet mass; and
kneading the resulting substantially homogeneous wet mass to substantially bulk material consistency.

2. The method of claim 1, wherein the at least one biopolymer is hyaluronic acid.

3. The method of claim 1, wherein the at least one pharmaceutically active ingredient is selected from the group consisting of one or more immunoglobulins, fragments or fractions of immunoglobulins, synthetic substance mimicking immunoglobulins or synthetic, semisynthetic or biosynthetic fragments or fractions thereof, chimeric, humanized or human monoclonal antibodies, Fab fragments, fusion proteins or receptor antagonists, antiangiogenic compounds, intracellular signaling inhibitors peptides having a molecular mass equal to or higher than 3 kDa, ribonucleic acids, desoxyribonucleic acids, plasmids, peptide nucleic acids, steroids, corticosteroids, an adrenocorticostatic, an antibiotic, an antidepressant, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug, an anticholinergic, an antihistaminic, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a haemostatic, a hormone and its synthetic or biosynthetic analogue, an immunosuppressant, an immunostimulant, a mitogen, a physiological or pharmacological inhibitor of mitogens, a mineralcorticoid, a muscle relaxant, a narcotic, a neurotransmitter, a precursor of a neurotransmitter, an oligonucleotide, a peptide, a (para)-sympathicomimetic, a (para)-sympatholytic, a protein, a sedating agent, a spasmolytic, a vasoconstrictor, a vasodilator, a vector, a virus, a virus-like particle, a virustatic, a wound-healing substance, and combinations thereof.

4. A method for manufacturing a biopolymeric bulk material, comprising:
providing at least a biopolymer microparticle dry powder comprising at least one biopolymer;
providing an aqueous solution;
optionally providing at least one pharmaceutically active ingredient;
mixing the biopolymer and aqueous solution by means of mechanical energy input to substantially homogeneous distribution; and
kneading the resulting substantially homogeneous wet mass to substantially bulk material consistency.

5. The method of claim 4, wherein the at least one biopolymer is hyaluronic acid.

6. The method of claim 4, wherein the at least one pharmaceutically active ingredient is selected from the group consisting of one or more immunoglobulins, fragments or fractions of immunoglobulins, synthetic substance mimicking immunoglobulins or synthetic, semisynthetic or biosynthetic fragments or fractions thereof, chimeric, humanized or human monoclonal antibodies, Fab fragments, fusion proteins or receptor antagonists, antiangiogenic compounds, intracellular signaling inhibitors peptides having a molecular mass equal to or higher than 3 kDa, ribonucleic acids, desoxyribonucleic acids, plasmids, peptide nucleic acids, steroids, corticosteroids, an adrenocorticostatic, an antibiotic, an antidepressant, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug, an anticholinergic, an antihistaminic, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a haemostatic, a hormone and its synthetic or biosynthetic analogue, an immunosuppressant, an immunostimulant, a mitogen, a physiological or pharmacological inhibitor of mitogens, a mineralcorticoid, a muscle relaxant, a narcotic, a neurotransmitter, a precursor of a neurotransmitter, an oligonucleotide, a peptide, a (para)-sympathicomimetic, a (para)-sympatholytic, a protein, a sedating agent, a spasmolytic, a vasoconstrictor, a vasodilator, a vector, a virus, a virus-like particle, a virustatic, a wound-healing substance, and combinations thereof.
